(19) United States
(12) Patent Application Publication
(10) Pub. No.: US 2025/0172511 A1
(43) Pub. Date: May 29, 2025

(54) MATERIAL DECOMPOSITION IN DUAL-ENERGY X-RAY IMAGING

(71) Applicant: Siemens Healthineers AG, Forchheim (DE)

(72) Inventor: Philipp Roser, Erlangen (DE)

(21) Appl. No.: 18/957,838

(22) Filed: Nov. 24, 2024

(30) Foreign Application Priority Data
Nov. 24, 2023 (EP) 23212063.4

(51) Int. Cl. G01N 23/04

(52) U.S. Cl. CPC G01N 23/04; G01N 2223/1016; G01N 2223/401; G01N 2223/405; G01N 2223/423

(57) ABSTRACT

In a computer-implemented method for material decomposition in dual-energy X-ray imaging, a first X-ray image dataset corresponding to a first X-ray energy spectrum, and a second X-ray image dataset corresponding to a second X-ray energy spectrum are obtained. At least one material-specific image dataset is generated by applying a decomposition module that contains a first sequence of processing steps or machine learning function to input data that depends on the first X-ray image dataset and the second X-ray image dataset. Before applying the decomposition module, a filter module and/or an artifact-reduction module is applied to the input data.

This application claims the benefit of European Patent Application No. EP 23212063.4, filed on Nov. 24, 2023, which is hereby incorporated by reference in its entirety.

### BACKGROUND

The present embodiments relate to a computer-implemented method for material decomposition in dual-energy X-ray imaging. The present embodiments also relate to a corresponding method for dual-energy X-ray imaging, and also to computer-implemented training methods, to a data processing apparatus, and to computer program products.

In dual-energy X-ray imaging (DE X-ray imaging), different X-ray image data is generated (e.g., two different X-ray projection images or two different three-dimensional volume reconstructions, using different X-ray energy spectra, such as using different radiation energies or radiation powers). For example, the term high-energy acquisition or high-energy scan and the term low-energy acquisition or low-energy scan are used here. The different X-ray energy spectra may be achieved by varying the operating parameters of the X-ray source (e.g., the operating voltage of the X-ray source) and/or by filters in the beam path (e.g., for beam hardening). Dual-energy X-ray imaging may be used to generate X-ray projection images but also for computed tomography (CT) (e.g., as a DECT method) or for cone-beam computed tomography (CBCT) (e.g., as a DE-CBCT method).

Since the attenuation coefficients of different materials (e.g., cerebrospinal fluid (CSF), blood, and X-ray selective contrast agents, such as contrast agents containing iodine or barium) vary by different degrees with varying radiation energies, then, from the different X-ray image data, based on different X-ray energy spectra, images or reconstructions in which a material (e.g., the contrast agent) is particularly enhanced may be generated, and further images in which the material is suppressed may be generated. This is referred to as material decomposition. For example, it is thereby possible to check whether significant bleeding has occurred during an intervention. For some symptoms, the contrast agent acquisition is crucial to being able to classify the findings.

For material decomposition, reference values for the attenuation of materials such as CSF or contrast agents may be defined for the different X-ray energy spectra. In combination with the actually measured attenuation values, it is then possible to perform the material decomposition largely using linear algebra methods. The material decomposition then provides material-specific image data as an output. Depending on the objective, on the usage, and on which materials are meant to be isolated, the material-specific image data may contain, for example, a contrast image, in which the contrast agent is enhanced, or a corresponding contrast reconstruction. The same is similarly possible for other materials such as water, fat, etc. Alternatively or additionally, the material-specific image data may contain a virtual non-contrast image (VNC image), in which the contrast agent is suppressed, or a corresponding VNC reconstruction.

Achieving material decomposition that is as accurate as possible may be fundamentally desirable not only so that a qualitative distinction may be made between the materials but also so that a quantitative analysis may be carried out (e.g., in order to be able to determine the specific amount of contrast agent). Effects that reduce the image quality include (e.g., for CBCT), for example, a comparatively low signal-to-noise ratio and/or contrast-to-noise ratio, scatter effects, cone beam artifacts, truncation, etc.

Heuristic or empirical methods are known that may improve the image quality (e.g., for CBCT methods), also then leading to improved image quality in the output from the material decomposition. However, this improved image quality primarily relates to visual perception by a human observer, but cannot improve the suitability for quantitative analysis, or only insufficiently.

In addition, machine-trained algorithms may be used for improving quality. As a result of their strong dependence on the training data used, however, such algorithms potentially have the problem of what is known as overfitting, and therefore, such algorithms also cannot improve the suitability for quantitative analysis, or only to a limited extent.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the quality of the material-specific image data in material decomposition in dual-energy X-ray imaging may be improved so as to facilitate, for example, quantitative material analysis or to facilitate improved quantitative material analysis.

The present embodiments are based on the idea of performing the material decomposition itself using a differentiable variable largely based on linear algebra methods, and by applying a trained function (e.g., a function trained by machine learning), the input data to which is X-ray image datasets corresponding to different X-ray energy spectra. According to one aspect of the present embodiments, a computer-implemented method is specified for material decomposition in dual-energy X-ray imaging. In this method, a first X-ray image dataset corresponding to a first X-ray energy spectrum, and a second X-ray image dataset corresponding to a second X-ray energy spectrum that differs from the first X-ray energy spectrum are obtained. At least one material-specific image dataset is generated by applying a decomposition module that contains a first function to input data that depends on the first X-ray image dataset and the second X-ray image dataset. Before applying the decomposition module, a filter module and/or an artifact-reduction module is applied to the input data.

The present embodiments are based on the finding that the quality of the material decomposition that may be achieved by the material decomposition module may be increased substantially if the X-ray image datasets undergo preprocessing by a filter module and/or an artifact-reduction module.

The actual material decomposition is based on applying linear algebra methods to a differentiable variable. A differentiable variable may be represented, for example, by plotting an attenuation value corresponding to the second X-ray energy spectrum over the attenuation value corresponding to the first X-ray energy spectrum, as explained in more detail in FIG. 2. The differentiability of the variable manifests itself in this plot by the fact that the attenuation values relating to specific materials are expressed in the slopes of the lines connecting the plotted points.

The actual material-decomposition algorithm is achieved by the first function and may be integrated as an adaptable or adapted differentiable sequence of processing steps or as a machine-learning function into any differentiable sequence of machine-learning processing steps. The differentiable material decomposition may be embedded in a machine-learning algorithm in order to facilitate task-based machine learning. This increases the robustness of the learned algorithm for the task of material decomposition based on X-ray image datasets that were acquired using different energy spectra. This makes it possible to calculate a loss term for particularly relevant materials. For example, the loss for neurological applications may be calculated in relation to an iodine map that is provided as a reference value.

Unless stated otherwise, all the acts of the computer-implemented method may be performed by a data processing apparatus that has at least one computing unit. For example, the at least one computing unit is configured or adapted to perform the acts of the computer-implemented method. For this purpose, the at least one computing unit may store, for example, a computer program containing commands that, on execution by the at least one computing unit, cause the at least one computing unit to execute the computer-implemented method.

However, an embodiment of a method that is not purely computer-implemented may be derived directly from each embodiment of the computer-implemented method by incorporating corresponding acts for generating the first X-ray image dataset and the second X-ray image dataset (e.g., by an X-ray source and an X-ray detector).

The first X-ray image dataset and the second X-ray image dataset each represent, from the same perspective, the same region of an object to be imaged. In the case of a C-arm X-ray device or an apparatus for CT or CBCT, the scan position of the X-ray source and X-ray detector with respect to each other and with respect to the object are therefore, for example, the same during generation of the first X-ray image dataset and the second X-ray image dataset.

The input data depending on the first X-ray image dataset and on the second X-ray image dataset may be understood, for example, to be that the input data includes the first X-ray image dataset and the second X-ray image dataset or respective preprocessed variants of the first X-ray image dataset and of the second X-ray image dataset. The preprocessing may include, for example, noise reduction, other artifact-reduction, image filtering, etc.

A material-specific image dataset may be understood to be, in the context of dual-energy X-ray imaging, for example, that in the material-specific image dataset a certain material is particularly enhanced or suppressed (e.g., in comparison with the first X-ray image dataset and second X-ray image dataset). What material is involved, for example, depends on the first function (e.g., on the parameterization of the first function) that, because of the differentiability of the first function, may itself likewise be trained, or depends on the training data used to train the first function, if it is a trained function. The material-specific image dataset may thus enhance or suppress, for example, fat, water, blood, CSF, contrast agent, etc. The material-specific image may also be a VNC image.

The first function may be, for example, a function trained using machine learning. For example, the first function may be an artificial neural network (ANN) (e.g., a convolutional neural network (CNN)), a vision transformer network, or a combination of a plurality of ANNs.

The first function may also include an optimizable or optimized function (e.g., a function that may be optimized, or has been optimized, by a gradient-based method). For example, a bilateral filter may be derived, and hence optimized, according to the associated parameters.

The decomposition module may also be part of a more complex algorithm that may include, in addition to the actual material decomposition, also other tasks (e.g., preprocessing, filtering, and/or three-dimensional reconstruction from a plurality of X-ray projection images, etc.). The first X-ray image dataset and the second X-ray image dataset may, for example, include X-ray projection images or three-dimensional reconstructions each based on a plurality of X-ray projection images.

The first X-ray energy spectrum and the second X-ray energy spectrum differ, for example, in terms of their radiation energy (e.g., photon energy) or radiation spectral energy distribution. A mean radiation energy of the first X-ray energy spectrum is, for example, higher than a mean radiation energy of the second X-ray energy spectrum. The different X-ray energy spectra may be achieved, for example, by different voltages (e.g., peak voltages) that are used to drive the X-ray source in order to generate the corresponding X-ray radiation. Optionally, filters may also be used additionally in the beam path (e.g., for beam hardening in the case of the X-ray energy spectrum having the higher mean radiation energy).

The method according to the present embodiments may achieve high-quality automatic material decomposition in dual-energy X-ray imaging (e.g., making a quantitative analysis of the material-specific image dataset possible even when the X-ray image datasets do not have optimum image quality, such as in the case of CBCT).

According to at least one embodiment, the first X-ray image dataset corresponds to a first X-ray projection image, and the second X-ray image dataset corresponds to a second X-ray projection image.

According to at least one embodiment, the first X-ray image dataset corresponds to a first reconstructed volume, and the second X-ray image dataset corresponds to a second reconstructed volume.

A reconstructed volume may be a three-dimensional reconstruction of the entire object or of a subregion of the object (e.g., of a three-dimensional slice of the object) based on X-ray projection images that were acquired from different directions (e.g., in a CT method or CBCT method or tomosynthesis method).

According to at least one embodiment, the at least one material-specific image dataset includes a contrast-agent image dataset.

In other words, the first X-ray image dataset and the second X-ray image dataset were generated after administration of a contrast agent, and therefore the contrast agent was in the object. The contrast agent may be a contrast agent containing iodine. The contrast-agent image dataset is then sometimes also referred to as an iodine image. The contrast agent may also be a contrast agent containing barium or another X-ray positive or X-ray negative contrast agent. This can be advantageous, for example, if concentrating the contrast agent in a region of the object is of particular interest.

According to at least one embodiment, the at least one material-specific image dataset includes a non-contrast image dataset (e.g., a VNC image dataset), so, for example, a VNC image or a three-dimensional VNC reconstruction.

In other words, the first X-ray image dataset and the second X-ray image dataset were again generated, for example, after administration of a contrast agent. In the non-contrast image dataset, the contrast agent is suppressed (e.g., the non-contrast image dataset simulates an acquisition that would be obtained without administration of a contrast agent). This may be advantageous, for example, if the contrast agent is present as an undesired, or no longer desired, residue.

According to at least one embodiment, an artifact-reduction module is applied before the decomposition module. An artifact-reduced first X-ray image dataset and artifact-reduced second X-ray image dataset are generated by applying an artifact-reduction module that contains at least one trained second function, to further input data, which depends on the first X-ray image dataset and the second X-ray image dataset (e.g., includes these). The input data depends on the artifact-reduced first X-ray image dataset and the artifact-reduced second X-ray image dataset (e.g., includes these).

The at least one second function may be, for example, at least one function trained using machine learning. For example, the at least one second function may include an ANN (e.g., a CNN or a vision transformer network, or a combination of a plurality of ANNs).

Depending on the embodiment, a trained second function (e.g., a trained ANN) of the at least one trained second function may be applied to the first X-ray image dataset and the second X-ray image dataset in order to generate the artifact-reduced first X-ray image dataset and the artifact-reduced second X-ray image dataset.

In other embodiments, a trained second function of the at least one trained second function may be applied to the first X-ray image dataset in order to generate the artifact-reduced first X-ray image dataset, and a trained further second function of the at least one trained second function may be applied to the second X-ray image dataset in order to generate the artifact-reduced second X-ray image dataset.

The at least one trained second function is trained, for example, to reduce artifacts in X-ray image datasets. The artifacts may include noise or other artifacts (e.g., resulting from scattered radiation or truncation, cone beam artifacts, etc.).

The quality of the input data for the decomposition module and consequently also the quality of the at least one material-specific image dataset are thereby increased.

A second function may also include an optimizable or optimized function (e.g., a function that may be optimized, or has been optimized, by a gradient-based method). For example, a bilateral filter may be derived, and hence optimized, according to the associated parameters.

The first function and the at least one second function may be trained together or separately from each other.

According to at least one embodiment, a filter module is applied before the decomposition module. A filtered first X-ray image dataset and a filtered second X-ray image dataset are generated by applying a filter module that contains at least one filter function to further input data that depends on the first X-ray image dataset and the second X-ray image dataset (e.g., contains these). The input data depends on the filtered first X-ray image dataset and the filtered second X-ray image dataset (e.g., contains these).

For example, the at least one filter function may be a function trained using machine learning. For example, the at least one second function may include an ANN (e.g., a CNN or a vision transformer network, or a combination of a plurality of ANNs). The at least one filter function may also be, however, an at least one conventional filter function that has not been trained using machine learning. In this case, parameters of the at least one filter function may be defined and/or be varied manually or automatically according to the usage case.

For example, the at least one filter function may be used for contrast improvement, for noise reduction, for edge improvement, etc.

Depending on the embodiment, a filter function of the at least one filter function may be applied to the first X-ray image dataset and the second X-ray image dataset in order to generate the filtered first X-ray image dataset and the filtered second X-ray image dataset. In other embodiments, a filter function of the at least one filter function may be applied to the first X-ray image dataset in order to generate the filtered first X-ray image dataset, and a further filter function of the at least one filter function may be applied to the second X-ray image dataset in order to generate the filtered second X-ray image dataset.

The quality of the input data for the decomposition module and consequently also the quality of the at least one material-specific image dataset are thereby increased.

Embodiments that provide for applying the filter module may also be combined with embodiments that provide for applying the artifact-reduction module. In this case, the artifact-reduction module and the filter module implement, for example, different noise reduction algorithms, or are not both used for noise reduction. For example, the artifact-reduction module may be applied to first further input data that depends on the first X-ray image dataset and the second X-ray image dataset (e.g., contains these) in order to generate the artifact-reduced first X-ray image dataset and the artifact-reduced second X-ray image dataset. The filter module may then be applied to second further input data that depends on the artifact-reduced first X-ray image dataset and the artifact-reduced second X-ray image dataset (e.g., contains these) in order to generate the filtered first X-ray image dataset and the filtered second X-ray image dataset. The reverse procedure may also be followed (e.g., the filter module is applied first and then the artifact-reduction module).

According to at least one embodiment, the at least one filter function contains at least one filter function for bilateral filtering and/or at least one filter function for differentiable guided filtering.

According to a further aspect of the present embodiments, a method for dual-energy X-ray imaging is specified. In this method, a first X-ray image dataset that represents an object to be imaged is generated by generating (e.g., using an X-ray source) first X-ray radiation corresponding to a first X-ray energy spectrum, and by detecting (e.g., using an X-ray detector) portions of the first X-ray radiation that pass through the object. A second X-ray image dataset that represents the object is generated by generating (e.g., using the X-ray source) second X-ray radiation corresponding to a second X-ray energy spectrum, and by detecting (e.g., using the X-ray detector) portions of the first X-ray radiation that pass through the object. A computer-implemented method according to the present embodiments for material decomposition is performed using the first X-ray image dataset and the second X-ray image dataset.

According to at least one embodiment of the method for dual-energy X-ray imaging, the method is carried out as a CT method.

According to at least one embodiment of the method for dual-energy X-ray imaging, the method is carried out as a CBCT method.

The present embodiments have an advantageous impact, for example, because of the generally lower image quality in CBCT methods.

According to at least one embodiment, the first X-ray image dataset corresponds to a first X-ray projection image, and the second X-ray image dataset corresponds to a second X-ray projection image. At least one reconstructed volume is generated based on the at least one material-specific image dataset.

Known reconstruction methods may be used to generate the reconstructed volume based on the at least one material-specific image dataset. For example, a number of material-specific image datasets are generated from different viewing directions, and the reconstructed volume is generated based on the number of material-specific datasets.

According to at least one embodiment, the first X-ray image dataset corresponds to a first reconstructed volume, and the second X-ray image dataset corresponds to a second reconstructed volume.

The first reconstructed volume and the second reconstructed volume may be generated using known reconstruction methods. For example, a number of X-ray projection images are generated from different viewing directions for both the first X-ray energy spectrum and the second X-ray energy spectrum, and the reconstructed volume is generated in each case based on the associated number of X-ray projection images.

According to a further aspect of the present embodiments, a computer-implemented training method is specified for providing a filter module and/or artifact-reduction module and a trained first function for use in a computer-implemented method according to the present embodiments for material decomposition in dual-energy X-ray imaging. A first X-ray training image dataset corresponding to a first X-ray energy spectrum and a second X-ray training image dataset corresponding to a second X-ray energy spectrum are obtained. At least one material-specific ground truth image dataset for the first X-ray training image dataset and the second X-ray training image dataset is obtained. At least one predicted material-specific image dataset is generated (e.g., predicted) by applying an artifact-reduction module or a filter module and an artifact-reduction module and then a decomposition module, which contains the untrained or part-trained first function, to input training data, which depends on the first X-ray training image dataset and the second X-ray training image dataset.

A defined loss function is evaluated, which depends on the at least one predicted material-specific image dataset and the at least one material-specific ground truth image dataset (e.g., on a deviation between the at least one predicted material-specific image dataset and the at least one material-specific ground truth image dataset). Parameters of the first function are updated depending on a result of the evaluation of the loss function (e.g., depending on a value of the loss function).

For example, the first function is a function that may be trained using machine learning (e.g., an ANN or a combination of a plurality of ANNs). The parameters of the first function then contain, for example, weighting factors and/or bias factors of the ANN or ANNs.

The acts of the training method may be repeated, for example, for a number of first and second X-ray training image datasets and associated ground truth image datasets until a defined break criterion or convergence criterion for the loss function is satisfied.

If the at least one predicted material-specific image dataset contains a plurality of predicted material-specific image datasets, then the at least one material-specific ground truth image dataset may include a material-specific ground truth image dataset for each predicted material-specific image dataset. Then, the loss function may include, for example, one loss term for each pair composed of a predicted material-specific image dataset and the associated material-specific ground truth image dataset.

According to at least one embodiment of the computer-implemented training method, this method is configured as a training method for providing the trained first function and at least one trained second function. A predicted artifact-reduced first X-ray image dataset and a predicted artifact-reduced second X-ray image dataset are generated by applying an artifact-reduction module that contains the at least one untrained or part-trained second function to further input training data that depends on the first X-ray training image dataset and the second X-ray training image dataset (e.g., includes these). The input training data depends on the predicted artifact-reduced first X-ray image dataset and the predicted artifact-reduced second X-ray image dataset (e.g., includes these).

According to at least one embodiment, parameters of the at least one second function are updated depending on a result of the evaluation of the loss function.

In such embodiments, the first function and the at least one second function are trained end to end, which requires less training data and leads to optimum harmonization in the training of the two tasks of artifact reduction and material decomposition.

For example, the at least one second function is at least one function that may be trained using machine learning (e.g., an ANN or a combination of a plurality of ANNs). The parameters of the at least one second function then contain, for example, weighting factors and/or bias factors of the ANN or ANNs.

According to at least one embodiment, an artifact-reduced first ground truth X-ray image dataset for the first X-ray training image dataset, and an artifact-reduced second ground truth X-ray image dataset for the second X-ray training image dataset are obtained. A defined further loss function is evaluated, which contains a loss term that depends on the predicted artifact-reduced first X-ray image dataset and the artifact-reduced first ground truth X-ray image dataset (e.g., depends on an associated deviation), and contains a further loss term that depends on the predicted artifact-reduced second X-ray image dataset and the artifact-reduced second ground truth X-ray image dataset (e.g., depends on an associated deviation). The parameters of the at least one second function are updated depending on a result of the evaluation of the further loss function.

The at least one second function is thus trained independently of the first function. This may be advantageous if the at least one second function is meant to be provided, for example, for different uses.

According to at least one embodiment of the computer-implemented training method, this method is configured as a training method for providing the trained first function and at least one filter function. A predicted filtered first X-ray image dataset and a predicted filtered second X-ray image dataset are generated by applying a filter module that contains the at least one filter function to further input training data that depends on the first X-ray training image dataset and the second X-ray training image dataset (e.g., contains these). The input training data depends on the predicted filtered first X-ray image dataset and the predicted filtered second X-ray image dataset.

According to at least one embodiment, at least one parameter of the at least one filter function is updated depending on a result of the evaluation of the loss function.

In such embodiments, the first function and the at least one filter function are trained end to end, which requires less training data and leads to optimum harmonization in the training of the two tasks of filtering and material decomposition.

For example, the at least one filter function may be at least one function that may be trained using machine learning (e.g., an ANN or a combination of a plurality of ANNs). The parameters of the at least one filter function then contain, for example, weighting factors and/or bias factors of the ANN or ANNs.

The at least one filter function may, however, also include a conventional filter algorithm (e.g., a bilateral filter). The parameters of the at least one filter function then contain, for example, a kernel size of a first filter kernel and/or a kernel size of a second filter kernel (e.g., a standard deviation in the case of a Gaussian filter kernel) and/or a window size of the filter.

According to at least one embodiment, a filtered first ground truth X-ray image dataset for the first X-ray training image dataset and a filtered second ground truth X-ray image dataset for the second X-ray training image dataset are obtained. A defined further loss function is evaluated, which contains a loss term that depends on the predicted filtered first X-ray image dataset and the filtered first ground truth X-ray image dataset (e.g., depends on an associated deviation), and which contains a further loss term that depends on the predicted filtered second X-ray image dataset and the filtered second ground truth X-ray image dataset (e.g., depends on an associated deviation). The parameters of the at least one filter function are updated depending on a result of the evaluation of the further loss function.

The at least one filter function is thus trained independently of the first function. This may be advantageous if the at least one filter function is meant to be provided, for example, for different uses.

According to a further aspect of the present embodiments, a data processing apparatus is specified. The data processing apparatus has at least one computing unit that is adapted to perform a computer-implemented method according to the present embodiments for material decomposition in dual-energy X-ray imaging and/or a computer-implemented training method according to the present embodiments.

A computing unit may be understood to be, for example, a data processing unit that contains a processing circuit. For example, the computing unit may thus process data for performing computing operations. These include also operations for performing indexed accesses to a data structure (e.g., to a look-up table (LUT)).

The computing unit may contain, for example, one or more computers, one or more microcontrollers, and/or one or more integrated circuits (e.g., one or more application-specific integrated circuits (ASIC), one or more field-programmable gate arrays (FPGA), and/or one or more systems on a chip (SoC)). The computing unit may also contain one or more processors (e.g., one or more microprocessors, one or more central processing units (CPU), one or more graphics processing units (GPU), and/or one or more signal processors, such as one or more digital signal processors (DSP)). The computing unit may also contain a physical or virtual interconnection of computers or other of the aforementioned units.

In various example embodiments, the computing unit contains one or more hardware and/or software interfaces and/or one or more memory units.

A memory unit may be configured as a volatile data storage medium (e.g., as a dynamic random access memory (DRAM) or a static random access memory (SRAM)) or as a non-volatile data storage medium (e.g., as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM)).

According to a further aspect of the present embodiments, an X-ray imaging apparatus is specified having a data processing apparatus according to the present embodiments, an X-ray source, an X-ray detector, and at least one control unit. The at least one control unit is configured to control the X-ray source to generate first X-ray radiation corresponding to a first X-ray energy spectrum. The X-ray detector is configured to generate the first X-ray image dataset, which represents an object to be imaged, and to do this by detecting portions of the first X-ray radiation that pass through the object. The at least one control unit is configured to control the X-ray source to generate second X-ray radiation corresponding to a second X-ray energy spectrum. The X-ray detector is configured to generate the second X-ray image dataset, which represents the object, and to do this by detecting portions of the second X-ray radiation that pass through the object.

The X-ray detector generating the first and second X-ray image datasets may be understood to be, for example, that the X-ray detector generates the first X-ray projection image as the first X-ray image dataset, and the second X-ray projection image as the second X-ray image dataset. If the X-ray image datasets are reconstructed volumes, the X-ray detector generates the corresponding X-ray projection images, and the at least one computing unit generates the X-ray image datasets.

The at least one control unit may be part of the at least one computing unit of the data processing apparatus, for example.

Further embodiments of the X-ray imaging apparatus according to the present embodiments follow directly from the various embodiments of the computer-implemented method according to the present embodiments and of the computer-implemented training method according to the present embodiments, and vice versa in each case. For example, individual features and associated explanations and advantages relating to the various embodiments for the method according to the present embodiments and for the training method according to the present embodiments may be applied analogously to corresponding embodiments of the X-ray imaging apparatus according to the present embodiments. For example, the X-ray imaging apparatus according to the present embodiments is configured or programmed to perform a computer-implemented method according to the present embodiments. For example, the X-ray imaging apparatus according to the present embodiments performs the computer-implemented method according to the present embodiments.

According to a further aspect of the present embodiments, a computer program containing commands is specified. When the commands are executed by a data processing apparatus, the commands cause the data processing apparatus to perform a computer-implemented method according to the present embodiments for material decomposition and/or a computer-implemented training method according to the present embodiments.

For example, the commands may exist as program code. The program code may be provided, for example, as binary code or assembler and/or as source code of a programming language (e.g., C) and/or as program script (e.g., Python).

According to a further aspect of the present embodiments, a further computer program containing further commands is specified. When the further commands are executed by an X-ray imaging apparatus according to the present embodiments (e.g., by the data processing apparatus of the X-ray imaging apparatus), the further commands cause the X-ray imaging apparatus to perform a method according to the present embodiments for dual-energy X-ray imaging.

For example, the further commands may exist as program code. The program code may be provided, for example, as binary code or assembler and/or as source code of a programming language (e.g., C) and/or as program script (e.g., Python).

According to a further aspect of the present embodiments, a computer-readable storage medium that stores a computer program according to the present embodiments and/or a further computer program according to the present embodiments is specified.

The computer program, the further computer program, and the computer-readable storage medium are each computer program products containing the commands and/or the further commands.

Further features and combinations of features of the present embodiments appear in the figures and the description of the figures and in the claims. For example, further embodiments need not necessarily contain all the features of one of the claims. Further embodiments may have features and combinations of features that are not mentioned in the claims.

The present embodiments are described in greater detail below with reference to specific example embodiments and associated schematic drawings. In the figures, same or functionally same elements may be denoted by the same reference signs. The description of same or functionally equivalent elements is not necessarily repeated when referring to different figures.

### BRIEF DESCRIPTION OF THE DRAWINGS

FIG. 1 shows a schematic representation of an example embodiment of an X-ray imaging apparatus;

FIG. 2 shows a schematic diagram illustrating the principle of three-material decomposition in dual-energy X-ray imaging;

FIG. 3 shows a schematic representation of an example embodiment of a computer-implemented method for material decomposition or of a corresponding computer-implemented training method;

FIG. 4 shows a schematic representation of a further example embodiment of a computer-implemented method for material decomposition or of a corresponding computer-implemented training method;

FIG. 5 shows a schematic representation of a further example embodiment of a computer-implemented method for material decomposition or of a corresponding computer-implemented training method;

FIG. 6 shows a schematic representation of a further example embodiment of a computer-implemented method for material decomposition or of a corresponding computer-implemented training method;

FIG. 7 shows a schematic representation of a further example embodiment of a computer-implemented method for material decomposition or of a corresponding computer-implemented training method;

FIG. 8 shows a schematic representation of a further example embodiment of a computer-implemented method for material decomposition or of a corresponding computer-implemented training method;

FIG. 9 shows a schematic representation of a further example embodiment of a computer-implemented method for material decomposition or of a corresponding computer-implemented training method;

FIG. 10 shows a schematic flow diagram of an example embodiment of a method for dual-energy imaging; and

FIG. 11 shows a schematic flow diagram of a further example embodiment of a method for dual-energy imaging.

### DETAILED DESCRIPTION

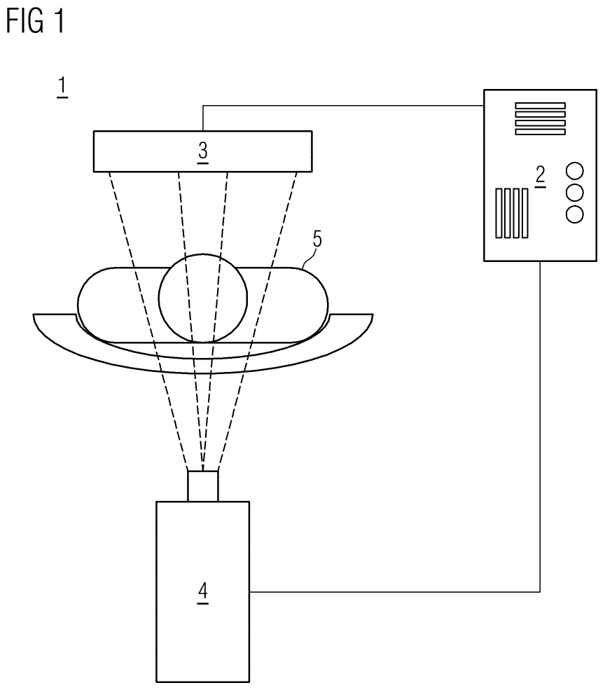

FIG. 1 shows a schematic representation of an example embodiment of an X-ray imaging apparatus 1 (e.g., for CBCT).

The X-ray imaging apparatus 1 has a data processing apparatus 2 according to the present embodiments, an X-ray source 4, an X-ray detector 3, and at least one control unit that may be part of the data processing apparatus 2, for example. The at least one control unit is configured to control the X-ray source 4 to generate first X-ray radiation corresponding to a first X-ray energy spectrum. The X-ray detector 3 is configured to generate first X-ray projection images, which represent an object 5 to be imaged, and to do this by detecting portions of the first X-ray radiation that pass through the object 5. The at least one control unit is configured to control the X-ray source 4 to generate second X-ray radiation corresponding to a second X-ray energy spectrum. The X-ray detector is configured to generate second X-ray projection images that represent the object 5, and to do this by detecting portions of the second X-ray radiation that pass through the object 5.

The data processing apparatus 2 has at least one computing unit that is configured to perform a computer-implemented method according to the present embodiments for material decomposition and a corresponding computer-implemented training method according to the present embodiments.

The data processing apparatus 2 uses a first X-ray projection image as a first X-ray image dataset, and a second X-ray projection image as a second X-ray image dataset, or generates, based on the first X-ray projection images, a first volume reconstruction 11, 21 as a first X-ray image dataset, and generates, based on the second X-ray projection images, a second volume reconstruction 12, 22 as a second X-ray image dataset.

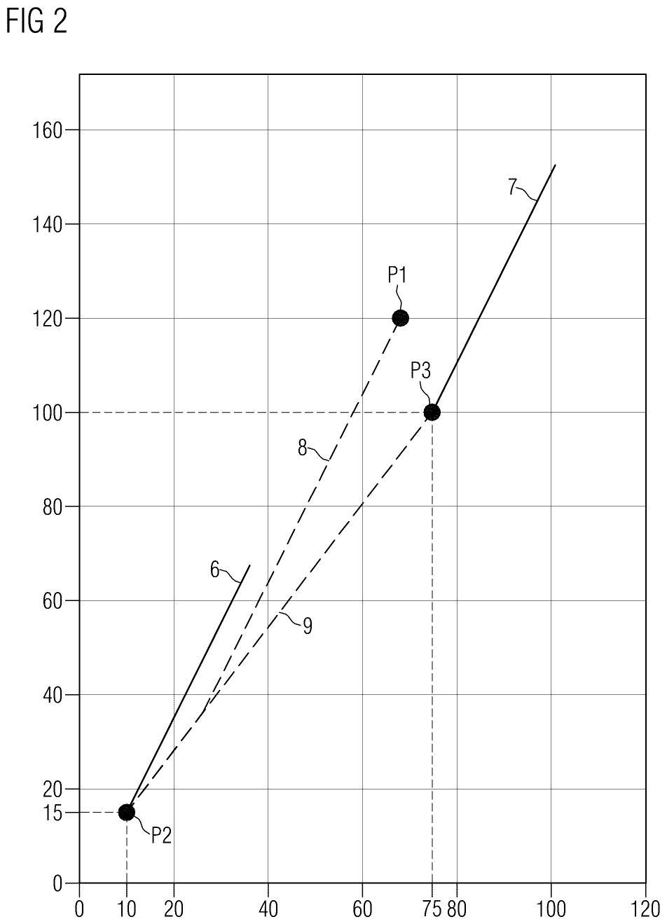

FIG. 2 shows a schematic diagram illustrating the principle of three-material decomposition in dual-energy X-ray imaging using a CT or CBCT method.

An attenuation value according to the first X-ray energy spectrum (e.g., corresponding to a tube voltage of the X-ray source 4 of 125 kV) is plotted on the horizontal axis. The attenuation value according to the second X-ray energy spectrum (e.g., corresponding to a tube voltage of the X-ray source 4 of 70 kV) is plotted on the vertical axis. For example, the attenuation values are given in Hounsfield units HU. Each voxel of a three-dimensional volume reconstruction may then be assigned a two-dimensional coordinate in the diagram of FIG. 2. In FIG. 2, the point P1 corresponds to the measured attenuation for a voxel, the point P2 corresponds to the attenuation by a first reference material (e.g., CSF), and the point P3 corresponds to the attenuation by a second reference material (e.g., a hemorrhage). An attenuation by iodine as a third reference material is not shown in FIG. 2 because the corresponding point P4 would lie at far higher values.

The line 6 emanates from point P2 and is parallel to the connecting line between P2 and P4. The line 7 emanates from point P3 and is parallel to the connecting line between P3 and P4. The line 8 emanates from point P1 and is parallel to the connecting line between P1 and P4. The line 9 connects the points P2 and P3. The line 8 intersects the line 9, and the length of the segment of the line 8 between the point P1 and the point of intersection with the line 9 may be interpreted as an approximate value of the attenuation for point P1 resulting from iodine. Thus, given points P1, P2, P3 and P4, it is possible to calculate, for each voxel, the attenuation caused by iodine, and to generate therefrom a corresponding contrast agent image or a contrast agent reconstruction. A similar process may also be followed for the other materials. The computer-implemented method according to the present embodiments for material decomposition may achieve, for example, the same results solely based on a trained first function.

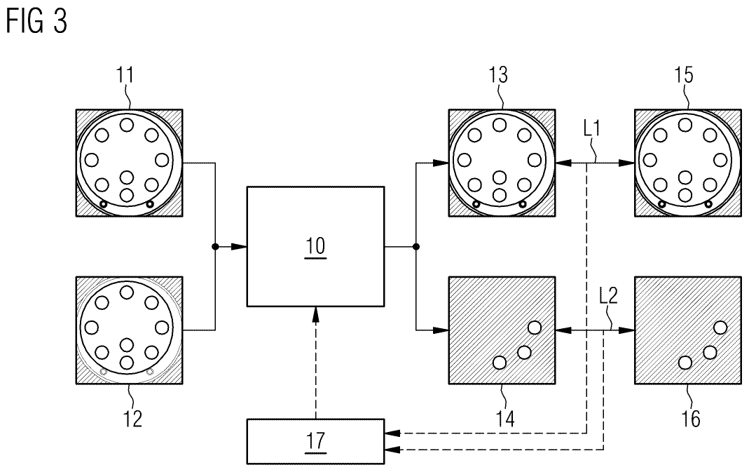

FIG. 3 shows a schematic representation of an example embodiment of a computer-implemented method according to the present embodiments for material decomposition or of a corresponding computer-implemented training method.

The decomposition module 10, which contains a first function, is applied to the first filtered, artifact-reduced, or filtered and artifact-reduced reconstructed volume reconstruction 11 and to the second filtered, artifact-reduced, or filtered and artifact-reduced reconstructed volume reconstruction 12, and on the basis thereof provides at least one material-specific volume reconstruction 13, 14 (e.g., an iodine reconstruction 14 and a VNC reconstruction 13). In this usage case, the first function is already trained, and hence, the computer-implemented method is completed, for example.

During the training of the first function, a loss function is evaluated depending on the at least one material-specific volume reconstruction 13, 14 and at least one material-specific ground truth volume reconstruction 15, 16, and parameters of the first function are updated by an optimization module 17 depending on a result of the evaluation of the loss function.

For example, a first loss term L1 is evaluated depending on the VNC reconstruction 13 and the associated ground truth VNC reconstruction 15, and a second loss term L2 is evaluated depending on the iodine reconstruction 14 and the associated ground truth iodine reconstruction 16. For example, the loss terms L1, L2 may be, or may be based on, known loss terms (e.g., a mean square error, a mean absolute error, a mean absolute percentage error, a structural similarity index, a histogram-based loss, etc.). For example, the loss function may correspond to a sum or weighted sum of the loss terms L1, L2.

The optimization module 17 may use known methods for updating the parameters of the first function (e.g., backpropagation).

Instead of the volume reconstructions 11, 12, the method may be carried out analogously based on corresponding X-ray projection images. Then, instead of the at least one material-specific volume reconstruction 13, 14, the result is at least one material-specific X-ray projection image.

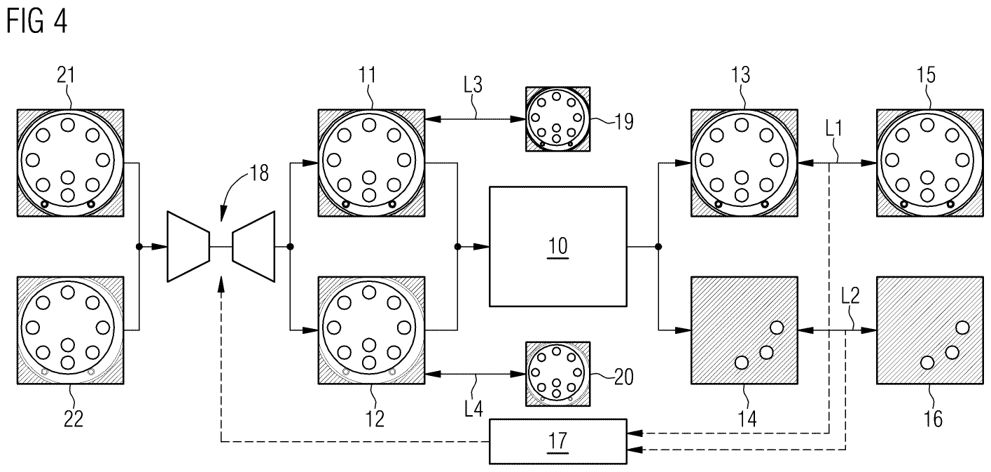

FIG. 4 shows a schematic representation of a further example embodiment of a computer-implemented method according to the present embodiments for material decomposition or of a corresponding computer-implemented training method, which are based on the embodiment of FIG. 3.

Here, the volume reconstructions 11, 12 are artifact-reduced volume reconstructions 11, 12. To achieve this, an artifact-reduction module that contains a second function 18 (e.g., a second ANN) is applied to original volume reconstructions 21, 22 corresponding to the first X-ray energy spectrum and the second X-ray energy spectrum, thereby providing the artifact-reduced volume reconstructions 11, 12. The remaining acts correspond to those explained with reference to FIG. 3. In this usage case, the second function is already trained, and hence, the computer-implemented method completed, for example.

During the training of the second function, a defined further loss function is evaluated. The further loss function contains a loss term L3 that depends on the artifact-reduced first volume reconstruction 11 and an artifact-reduced first ground truth volume reconstruction 19, and contains a further loss term L4 that depends on the predicted artifact-reduced second volume reconstruction 12 and an artifact-reduced second ground truth volume reconstruction 20. Parameters of the second function 18 are updated depending on a result of the evaluation of the further loss function by the optimization module 17.

For example, the loss terms L3, L4 may be, or may be based on, known loss terms (e.g., a mean square error, a mean absolute error, a mean absolute percentage error, a structural similarity index, a histogram-based loss, etc.). For example, the further loss function may correspond to a sum or weighted sum of the loss terms L3, L4.

The optimization module 17 may use known methods for updating the parameters of the first function (e.g., backpropagation). For example, the optimization module 17 may train the first function and the second function 18 separately from each other.

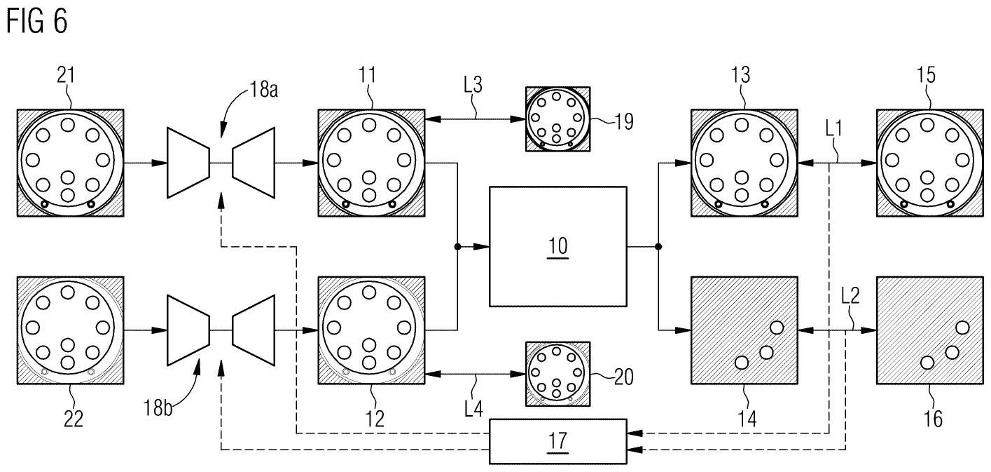

In some embodiments, the second function 18 is replaced by a second function 18a that is applied to the original volume reconstructions 21 and provides the artifact-reduced volume reconstructions 11, and by a further second function 18b that is applied to the original volume reconstructions 22 and provides the artifact-reduced volume reconstructions 12. FIG. 6 shows such a method schematically.

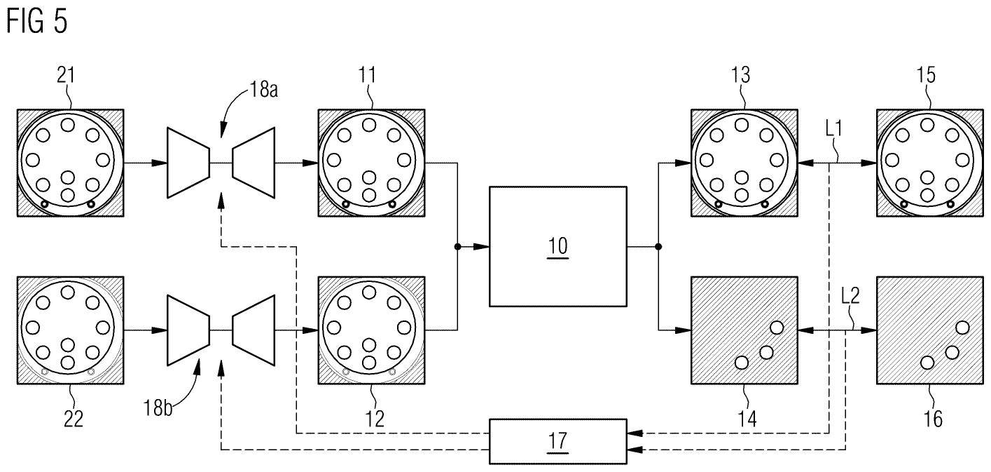

Alternative embodiments dispense with the further loss function, and the first function and the second function 18 are trained jointly end to end based on the loss function containing the loss terms L1, L2. The ground truth volume reconstructions 19, 20 are then not required. Also, such embodiments may be combined with embodiments in which the second function 18 is replaced by the second functions 18a, 18b, as shown schematically in FIG. 5.

Instead of the reconstructed volumes 11, 12, the method may be carried out analogously based on corresponding X-ray projection images. Then, instead of the at least one material-specific volume reconstruction 13, 14, the result is at least one material-specific X-ray projection image.

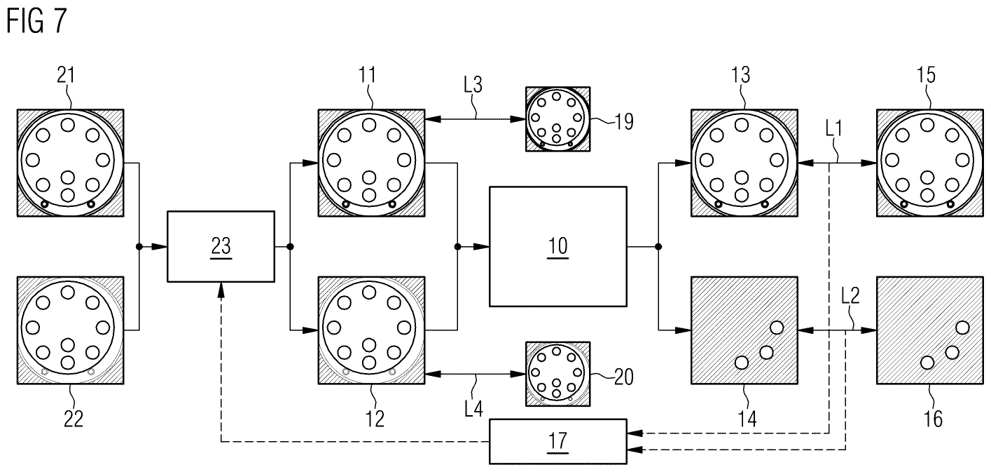

FIG. 7 shows a schematic representation of a further example embodiment of a computer-implemented method according to the present embodiments for material decomposition or of a corresponding computer-implemented training method, which are based on the embodiment of FIG. 4. In the embodiment of FIG. 7, the second function 18 is replaced by a shared filter function 23 (e.g., a shared bilateral filter). The filter function 23 is not necessarily embodied as an ANN; its parameters (e.g., kernel sizes and/or a window size of the filter), however, may likewise be trained separately from the first function based on the loss terms L3, L4, or may be trained end to end with the first function.

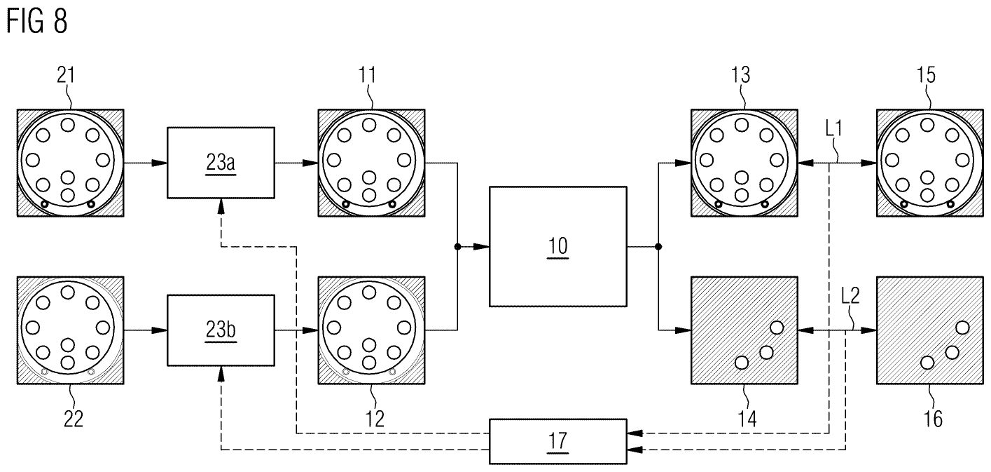

FIG. 8 shows a schematic representation of a further example embodiment of a computer-implemented method according to the present embodiments for material decomposition or of a corresponding computer-implemented training method, which is based on the embodiment of FIG. 7. Here, the shared filter function 23 is replaced by two individual filter functions 23a, 23b that are applied to the original volume reconstructions 21 and to the original volume reconstructions 22, respectively. The training of the filter functions 23a, 23b is performed end to end with the first function, as explained with reference to FIG. 5.

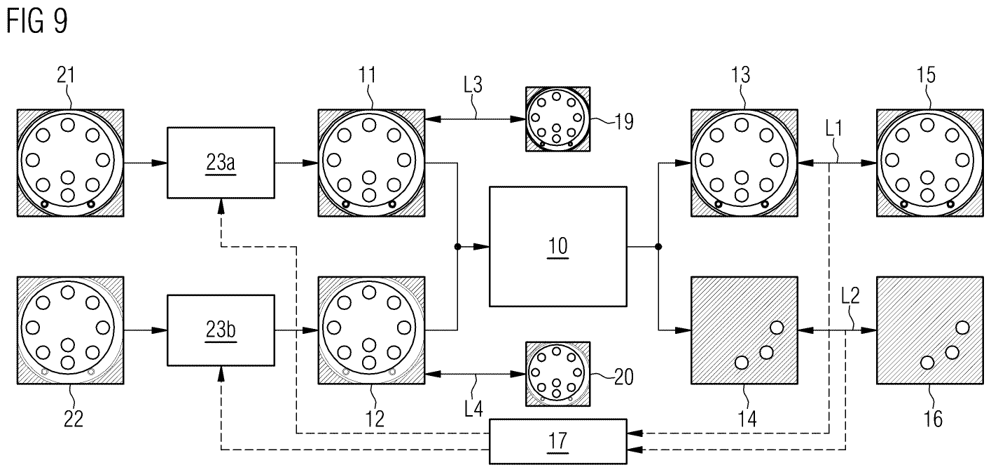

FIG. 9 shows a schematic representation of a further example embodiment of a computer-implemented method according to the present embodiments for material decomposition or of a corresponding computer-implemented training method, which are based on the embodiment of FIG. 7. Here, the shared filter function 23 is replaced by two individual filter functions 23a, 23b that are applied to the original volume reconstructions 21 and to the original volume reconstructions 22, respectively. The training of the filter functions 23a, 23b is performed separately from the first function, as explained with reference to FIG. 6.

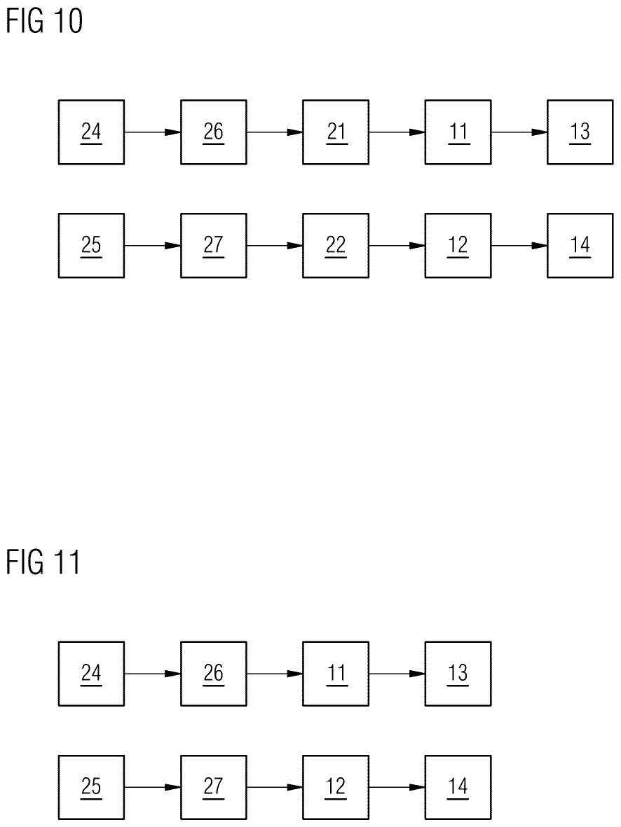

FIG. 10 shows a schematic flow diagram of an example embodiment of a method according to the present embodiments for dual-energy imaging using a computer-implemented method according to the present embodiments for material decomposition.

A number of first X-ray projection images 24 are generated for the first X-ray energy spectrum. A number of artifact-reduced and/or filtered first X-ray projection images 26 are generated on the basis thereof. The first volume reconstruction 21 is generated on the basis thereof and processed further as explained above with reference to FIG. 4 to FIG. 9.

A number of second X-ray projection images 25 are generated for the second X-ray energy spectrum. A number of artifact-reduced and/or filtered second X-ray projection images 27 are generated on the basis thereof. The second volume reconstruction 22 is generated on the basis thereof and processed further as explained above with reference to FIG. 4 to FIG. 9.

FIG. 11 shows a schematic flow diagram of a further example embodiment of a method according to the present embodiments for dual-energy imaging using a computer-implemented method according to the present embodiments for material decomposition.

The number of first X-ray projection images 24 are generated for the first X-ray energy spectrum. The number of artifact-reduced and/or filtered first X-ray projection images 26 are generated on the basis thereof. The first volume reconstruction 11 is generated on the basis thereof and processed further as explained above with reference to FIG. 3.

The number of second X-ray projection images 25 are generated for the second X-ray energy spectrum. The number of artifact-reduced and/or filtered second X-ray projection images 27 are generated on the basis thereof. The second volume reconstruction 12 is generated on the basis thereof and processed further as explained above with reference to FIG. 3.

As described (e.g., with reference to the figures), the present embodiments make it possible to improve the quality of the material-specific image data in material decomposition in dual-energy X-ray imaging so as to facilitate, for example, improved quantitative material analysis.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for material decomposition in dual-energy X-ray imaging, the computer-implemented method comprising:
obtaining a first X-ray image dataset corresponding to a first X-ray energy spectrum, and a second X-ray image dataset corresponding to a second X-ray energy spectrum; and
generating at least one material-specific image dataset, the generating of the at least one material-specific image dataset comprising applying a decomposition module that contains a first sequence of processing steps or machine learning function to input data that depends on the first X-ray image dataset and the second X-ray image dataset,
wherein, before applying the decomposition module, a filter module, an artifact-reduction module, or the filter module and the artifact-reduction module are applied to the input data.

2. The computer-implemented method of claim 1, wherein the first X-ray image dataset corresponds to a first X-ray projection image, and the second X-ray image dataset corresponds to a second X-ray projection image, or
wherein the first X-ray image dataset corresponds to a first reconstructed volume, and the second X-ray image dataset corresponds to a second reconstructed volume.

3. The computer-implemented method of claim 1, wherein the at least one material-specific image dataset includes a contrast-agent image dataset, a virtual non-contrast image dataset, or the contrast-agent image dataset and the virtual non-contrast image dataset.

4. The computer-implemented method of claim 1, further comprising:
generating an artifact-reduced first X-ray image dataset and an artifact-reduced second X-ray image dataset, the generating of the artifact-reduced first X-ray image dataset and the artifact-reduced second X-ray image dataset comprising applying an artifact-reduction module that contains at least one trained second function to further input data that depends on the first X-ray image dataset and the second X-ray image dataset,
wherein the input data depends on the artifact-reduced first X-ray image dataset and the artifact-reduced second X-ray image dataset.

5. The computer-implemented method of claim 1, further comprising:
generating a filtered first X-ray image dataset and a filtered second X-ray image dataset, the generating of the filtered first X-ray image dataset and the filtered second X-ray image dataset comprising applying a filter module that contains at least one filter function to further input data that depends on the first X-ray image dataset and the second X-ray image dataset,
wherein the input data depends on the filtered first X-ray image dataset and the filtered second X-ray image dataset.

6. The computer-implemented method of claim 5, wherein the at least one filter function contains at least one filter function for bilateral filtering, at least one filter function for differentiable guided filtering, and the at least one filter function for bilateral filtering and the at least one filter function for differentiable guided filtering.

7. A method for dual-energy X-ray imaging, the method comprising:
generating a first X-ray image dataset that represents an object to be imaged, the generating of the first X-ray image comprising:
generating first X-ray radiation corresponding to a first X-ray energy spectrum; and
detecting portions of the first X-ray radiation that pass through the object;
generating a second X-ray image dataset that represents the object, the generating of the second X-ray image comprising:
generating second X-ray radiation corresponding to a second X-ray energy spectrum; and
detecting portions of the first X-ray radiation that pass through the object; and
performing a computer-implemented method for material decomposition in dual-energy X-ray imaging, the computer-implemented method comprising:
generating at least one material-specific image dataset, the generating of the at least one material-specific image dataset comprising applying a decomposition module that contains a first sequence of processing steps or machine learning function to input data that depends on the first X-ray image dataset and the second X-ray image dataset,
wherein, before applying the decomposition module, a filter module, an artifact-reduction module, or the filter module and the artifact-reduction module are applied to the input data.

8. The method of claim 7, wherein the method is carried out as a computed tomography method or as a cone-beam computed tomography method, and
wherein:
the first X-ray image dataset corresponds to a first X-ray projection image, the second X-ray image dataset corresponds to a second X-ray projection image, and at least one reconstructed volume is generated based on the at least one material-specific image dataset; or
the first X-ray image dataset corresponds to a first reconstructed volume, and the second X-ray image dataset corresponds to a second reconstructed volume.

9. A computer-implemented training method for providing a trained first function for use in a computer-implemented method, the computer-implemented training method comprising:
obtaining a first X-ray training image dataset corresponding to a first X-ray energy spectrum, and a second X-ray training image dataset corresponding to a second X-ray energy spectrum;
obtaining at least one material-specific ground truth image dataset for the first X-ray training image dataset and the second X-ray training image dataset;
generating at least one predicted material-specific image dataset, the generating of the at least one predicted material-specific image dataset comprising applying a decomposition module that contains a first sequence of processing steps or machine learning function to input training data that depends on the first X-ray training image dataset and the second X-ray training image dataset;
applying, before applying the decomposition module, a filter module, an artifact-reduction module, or the filter module and the artifact-reduction module to the input data;
evaluating a defined loss function that depends on the at least one predicted material-specific image dataset and the at least one material-specific ground truth image dataset; and
updating parameters of the first function depending on a result of the evaluating of the defined loss function.

10. The computer-implemented training method of claim 9, further comprising:
obtaining an artifact-reduced first ground truth X-ray image dataset for the first X-ray training image dataset and an artifact-reduced second ground truth X-ray image dataset for the second X-ray training image dataset;
generating a predicted artifact-reduced first X-ray image dataset and a predicted artifact-reduced second X-ray image dataset, the generating of the predicted artifact-reduced first X-ray image dataset and the predicted artifact-reduced second X-ray image dataset comprising applying an artifact-reduction module that contains at least one untrained or part-trained second function to further input training data that depends on the first X-ray training image dataset and the second X-ray training image dataset, wherein the input training data depends on the predicted artifact-reduced first X-ray image dataset and the predicted artifact-reduced second X-ray image dataset;
evaluating a defined further loss function that contains a loss term that depends on the predicted artifact-reduced first X-ray image dataset and the artifact-reduced first ground truth X-ray image dataset, and contains a further loss term that depends on the predicted artifact-reduced second X-ray image dataset and the artifact-reduced second ground truth X-ray image dataset; and
updating parameters of the at least one second function depending on a result of the evaluation of the further loss function.

11. The computer-implemented training method of claim 9, further comprising:
obtaining a filtered first ground truth X-ray image dataset for the first X-ray training image dataset and a filtered second ground truth X-ray image dataset for the second X-ray training image dataset;
generating a predicted filtered first X-ray image dataset and a predicted filtered second X-ray image dataset, the generating of the predicted filtered first X-ray image dataset and the predicted filtered second X-ray image dataset comprising applying a filter module that contains the at least one filter function to further input training data that depends on the first X-ray training image dataset and the second X-ray training image dataset, wherein the input training data depends on the predicted filtered first X-ray image dataset and the predicted filtered second X-ray image dataset;
evaluating a defined further loss function that contains a loss term that depends on the predicted filtered first X-ray image dataset and the filtered ground truth X-ray image dataset, and contains a further loss term that depends on the predicted filtered second X-ray image dataset and the filtered second ground truth X-ray image dataset; and
updating parameters of the at least one filter function depending on a result of the evaluating of the further loss function.

12. A computer-implemented training method for providing a trained first function and at least one trained second function for use in a computer-implemented method, the computer-implemented training method comprising:
providing a trained first function for use in the computer-implemented method, the providing of the trained first function comprising:
obtaining a first X-ray training image dataset corresponding to a first X-ray energy spectrum, and a second X-ray training image dataset corresponding to a second X-ray energy spectrum;
obtaining at least one material-specific ground truth image dataset for the first X-ray training image dataset and the second X-ray training image dataset;
generating at least one predicted material-specific image dataset, the generating of the at least one predicted material-specific image dataset comprising applying a decomposition module that contains a first sequence of processing steps or machine learning function to input training data that depends on the first X-ray training image dataset and the second X-ray training image dataset;
applying, before applying the decomposition module, a filter module, an artifact-reduction module, or the filter module and the artifact-reduction module to the input data;
evaluating a defined loss function that depends on the at least one predicted material-specific image dataset and the at least one material-specific ground truth image dataset; and
updating parameters of the first function depending on a result of the evaluating of the defined loss function;
generating a predicted artifact-reduced first X-ray image dataset and a predicted artifact-reduced second X-ray image dataset, the generating of the predicted artifact-reduced first X-ray image and the predicted artifact-reduced second X-ray image dataset comprising applying an artifact-reduction module that contains at least one untrained or part-trained second function to further input training data that depends on the first X-ray training image dataset and the second X-ray training image dataset, wherein the input training data depends on the predicted artifact-reduced first X-ray image dataset and the predicted artifact-reduced second X-ray image dataset; and
updating parameters of the at least one second function depending on a result of the evaluating of the defined loss function.

13. An X-ray imaging apparatus comprising:
a data processing apparatus comprising:
a processor configured for material decomposition in dual-energy X-ray imaging, the processor being configured for material decomposition in dual-energy X-ray imaging comprising the processor being configured to:
obtain a first X-ray image dataset corresponding to a first X-ray energy spectrum, and a second X-ray image dataset corresponding to a second X-ray energy spectrum; and
generate at least one material-specific image dataset, the generation of the at least one material-specific image dataset comprising application of a decomposition module that contains a first sequence of processing steps or machine learning function to input data that depends on the first X-ray image dataset and the second X-ray image dataset, wherein, before the application of the decomposition module, a filter module, an artifact-reduction module, or the filter module and the artifact-reduction module are applied to the input data;
an X-ray source;
an X-ray detector; and
at least one control unit configured to:
control the X-ray source to generate first X-ray radiation corresponding to the first X-ray energy spectrum; and
control the X-ray source to generate second X-ray radiation corresponding to the second X-ray energy spectrum,
wherein the X-ray detector is configured to:
generate the first X-ray image dataset, which represents an object to be imaged, the generation of the first X-ray image dataset comprising detection of portions of the first X-ray radiation that pass through the object; and
generate the second X-ray image dataset, which represents the object to be imaged, the generation of the second X-ray image dataset comprising detection of portions of the second X-ray radiation that pass through the object.
